(19) United States
(12) Patent Application Publication
(10) Pub. No.: US 2005/0089555 A1
(43) Pub. Date: Apr. 28, 2005

(54) Medicinal targeted local lipolysis

(75) Inventors: Peter Boderke, Frankfurt (DE); Rainer Pooth, Dreiech-Goetzenhain (DE); Juergen Sandow, Glashuetten (DE); Matthias Gossel, Hofheim (DE); Karl-Heinz Nietsch, Neuss (DE); Gerhard Sattler, Darmstadt (DE); Gerhard Vogel, Aalen (DE)

Correspondence Address: ROSS J. OEHLER;AVENTIS PHARMACEUTICALS INC., ROUTE 202-206, MAIL CODE: D303A, BRIDGEWATER, NJ, 08807, US

(73) Assignee: Aventis Pharma Deutschland GmbH, Frankfurt am Main (DE)

(21) Appl. No.: 10/970,815

(22) Filed: Oct. 21, 2004

Related U.S. Application Data
(60) Provisional application No. 60/551,142, filed on Mar. 8, 2004.

(30) Foreign Application Priority Data
Oct. 24, 2003 (DE) 10349979.2

(51) Int. Cl. A61K 31/685; A61K 31/56; A61K 9/127

(52) U.S. Cl. 424/450; 514/78; 514/171

(57) ABSTRACT

Aqueous phospholipid systems comprising at least one phospholipid, at least one bile acid and water are suitable for producing medicaments for the treatment of adipose tissue disorders and lead to regression of the pathologically proliferated adipose tissue.

### CROSS-REFERENCE TO RELATED APPLICATIONS

This application claims the benefit of U.S. Provisional Application No. 60/551,142, filed Mar. 8, 2004, and incorporated herein by reference.

### DESCRIPTION OF THE INVENTION

The invention relates to aqueous phospholipid systems comprising at least one phospholipid, at least one bile acid and water, which are suitable for producing medicaments for the treatment of adipose tissue disorders and for regression of pathologically proliferated adipose tissue.

Aqueous phospholipidic phospholipid systems are known for various applications. Thus, these systems are employed for example in the cosmetic sector or for producing pharmaceutical products. These systems are distinguished by having spherical vesicles which are also referred to as liposomes. The boundary of said liposomes to the outside is formed by a lipid bilayer membrane, and they contain an aqueous phase inside. Aqueous phospholipid systems comprising at least one phospholipid, at least one bile acid and water are described for example in U.S. Pat. No. 6,663,885.

A marketed product is Lipostabil® N i.V. (Rote Liste, March 2003) which constitutes an aqueous phospholipid system which comprises phospholipids, bile acid, DL-alpha-tocopherol, ethanol and water and is approved for the prophylaxis and treatment of fat embolisms.

It is reported that fat pads like those occurring in overweight people underneath the eyes, on the abdomen or on the hips shrink, and esthetic improvements in the appearance of the treated people are said to occur when these people received subcutaneous injection of Lipostabil® N i.V. (Patricia Guedes Rittes, The Use of Phosphatidylcholine for Correction of Lower Lid Bulging Due to Prominent Fat Pads, Dermatol Surg. 2001; 27: 391-392).

With the aim of finding effective compounds for the treatment of disorders of adipose tissue, it has now been found that the liposome system employed according to the invention leads to a regression in pathologically proliferated adipose tissue. Lipolysis of the adipose tissue takes place, and the relevant pathologically proliferated adipose tissue region recedes. As stated below, these disorders involve not just esthetically upsetting proliferations of adipose tissue, but painful conditions and impairments of body functions.

The invention therefore relates to the use of aqueous phospholipid systems comprising
a) at least one phospholipid,
b) at least one bile acid, and
c) water
   for producing a medicament for the treatment of adipose tissue disorders.

The invention further relates to the use of said aqueous liposome system for producing a medicament for regression of adipose tissue tumors.

The term “phospholipid” means compounds such as 3-sn-phosphatidylcholine, soya (Phospholipon 90), 3-sn-phosphatidylcholine, reduced soya (Phospholipon 90H), 3-(3sn)-phospohatidyl)glycerol soya (Phospholipon G), dimyristoylphosphatidylglycerol, lyso-phosphatidylcholine or dipalmitoylphosphatidylglycerol and the physiologically tolerated salts thereof.

The term “bile acid” means compounds such as deoxycholic acid, cholic acid, lithocholic acid, chenodeocycholic acid, hyodeoxycholic acid, trihydroxycoprostanic acid, ursodeoxycholic acid, taurocholic acid or glycocholic acid, and the physiologically tolerated salts thereof.

The term “adipose tissue disorders” means for example the following disorders: Lipomas are adipose tissue tumors, which are benign, slow-growing, usually spherical, possibly pedunculated (=I. pendulum) or even villous (=I. arborescens, for example of the synovial villi) mesenchymal tumors composed of—enlarged—adipose tissue cells, preferentially in a subcutaneous cell tissue, possibly with central ossification (=I. ossificans), becoming mucoid (=I. myxomatodes) or calcifying (=I. petrificans), also with increased connective tissue and capsule formation (=I. fibrosum), neoangiogenesis (=I. teleangiectodes), rarely showing malignant degeneration (=I. sarcomatodes, liposarcoma). They are to be categorized as pathological because they grow and their connective tissue envelope may be painful per se, as well as the compression derived therefrom on blood vessels, which may cause neuralgia.

Dercum's disease, called lipomatosis dolorosa, is a special type of hypertrophic proliferation of adipose tissue, which is located between the dermal fat fascia (Kampa's fat fascia) and the underside of the dermis. Hormonal effects lead to an enhanced water-binding capacity of these fat cells which themselves in turn bring about, through pressure phenomena, lymph tract obstructions in the region of the initial fern-like lymph vessels and with which additional compressive and irritant effects are exerted on the peripheral sensory nerves, so that these patients display an extremely painful sensitivity to touch. Over the course of several years up to decades there is formation of irregular fatty nodules in disseminated locations underneath the dermis, which becomes thinner during the aging process, some of which nodules have painful and highly dysesthetic characteristics.

Madelung's neck (Lanois-Bensaude syndrome) is an adipose tissue inflammation with adipose tissue proliferation in which a dystrophic adipose tissue tumor formation is accompanied by subcutaneous scar-like connective tissue compaction. In such cases, surgical procedures can often be only partially successful, because essential anatomic structures are involved in this process and the disorder is manifested essentially in the region of the head, neck and shoulders.

Lipedema is a painful adipose tissue swelling which occurs especially on the lower legs of women and shows a progressive course and characteristics with increasing age.

Piezogenic nodules are nodules on the edges of the hands and the heels which are caused by pressure and occur as multiple adipose tissue hernias, mainly in the medial region of the heel in obese people. They are usually defects in the septation of the subcutaneous adipose tissue which are regarded by patients as cosmetically or functionally disturbing.

Xanthelasma is a pale yellow, slightly raised plaque-like deposit of cholesterol in the region of the eyelids. They are soft and easily displaceable and usually occur symmetrically on both eyes. It is caused by local derangements of lipid metabolism. Postmenopausal women are affected particularly frequently. Diabetes mellitus and elevated blood liquid levels are also associated with an increased risk of developing it. Xanthelasmas may cause psychological stress because of their appearance.

Various types of lipodystrophy, such as lipodystrophy syndrome which may occur in HIV patients after treatment with protease inhibitors, dystrophia adiposogenitalis, which is an endocrine disorder in adolescent girls, sphingolipidoses, which usually have hereditary characteristics, such as angiokeratoma corpis (Fabry's syndrome) or gangliosidoses with cutaneous manifestations.

The term “regression” means the lipolysis of the adipose tissue and regression of the proliferated adipose region.

The abovementioned adipose tissue disorders show, in contrast to the food-related obesity-correlated lipohypertrophy, tissue conditions or identities which can be pathologically differentiated unambiguously and which can be described by histological parameters of scarring and inflammation, but also by connective tissue encapsulations and by changes in the histological adipose tissue morphology itself.

The invention therefore relates to the use of aqueous phospholipid systems comprising
a) at least one phospholipid,
b) at least one bile acid, and
c) water
   for producing a medicament for the treatment of cellulite.

Cellulite is a special type of hypertrophic proliferation of adipose tissue, which is located between the dermal fat fascia (Kampa's fat fascia) and the underside of the dermis. Hormonal effects lead to an enhanced water-binding capacity of these fat cells which themselves in turn bring about, through pressure phenomena, lymph tract obstructions in the region of the initial fern-like lymph vessels. Over the course of several years up to decades there is formation of irregular fatty nodules in disseminated locations underneath the dermis, which becomes thinner during the aging process, some of which nodules have painful and highly dysesthetic characteristics.

The invention also relates to the use of at least one phospholipid or at least one bile acid for producing a medicament for the treatment of adipose tissue disorders or cellulite.

If only phospholipid or bile acid is employed alone, the same conditions and definitions apply as for the abovementioned mixtures of phospholipid and bile acid.

The invention also relates to the use of phospholipid in which the phospholipid is in the form of a physiologically tolerated salt, for example as sodium, potassium and/or ammonium salt.

The phospholipid can be isolated from oil seeds, rapeseed, soybean or sunflowers and, after appropriate application, be employed in the liposome system. Lecithin, for example from chicken egg, is also suitable. Phospholipids from soybeans are preferred. The invention also relates to the use of phospholipid in which the phospholipid is the phosphatidylcholine from soybean and is isolated therefrom. Especially when the phospholipid consists of at least 90 percent by weight (% by weight) of soybean phosphatidylcholine, in particular 95% by weight.

The invention also relates to the use of a bile acid in which the bile acid is in the form of a physiologically tolerated salt. This may be for example a sodium, potassium and/or ammonium salt of deoxycholic acid, cholic acid, lithocholic acid, chenodeocycholic acid, hyodeoxycholic acid, trihydroxycoprostannic acid, ursodeoxycholic acid, taurocholic acid or glycocholic acid.

The mass ratio of phospholipid to bile acid is, in % by weight, from 30:1 to 1:0.03, preferably from 1:0.7 to 1:0.1, in particular 1:0.6 to 1:0.3.

The phospholipid concentration in the liposome system is from 0.5% by weight to 30% by weight, preferably from 5% by weight to 25% by weight, in particular from 10% by weight to 20% by weight. The liposomes have a diameter of from 30 nm to 180 nm, preferably from 30 nm to 130 nm, in particular from 50 nm to 90 nm. These liposomes can be sterilized by filtration without difficulty, employing filters with a pore diameter of 0.2 μm. The pH of the liposome system is around the neutral point, preferably from 5.0 to 8.0, in particular from 6.2 to 7.4.

The liposome system is produced for example by dissolving or dispersing at least one phospholipid and at least one bile acid in the abovementioned ratio to one another in an organic solvent. This solution or dispersion is then concentrated, and thereafter water is added to form the liposome system. Production of the liposome system can be promoted after addition of the water by extrusion, high-pressure homogenization and/or ultrasound treatment. The treatment takes place below 40° C., preferably from 20° C. to 30° C. Suitable organic solvents are ethanol, propanol, isopropyl alcohol or benzyl alcohol, in each case alone or in a mixture. The volumes of alcohols remaining after concentration should be from 0 percent by volume (% by volume) to 20% by volume, preferably from 0% by volume to 10% by volume. Processes for producing the liposome systems are also described in European patent application EP 0 470 437 or EP 0 615 746.

The liposome system employed according to the invention is administered by subcutaneous, intra-articular, intraperitoneal, intramuscular or intravenous injection. Subcutaneous injection is preferred. Also possible is percutaneous administration in various carrier media and with use of various aids, for example iontophoresis.

Uniform introduction of the liposome system employed according to the invention should take place by a tumescent method which makes use of the hydrostatic pressure in order to ensure uniform distribution.

Suitable formulations are, for example, suspensions, emulsions or injectable solutions, and products with protracted release of active ingredient, in the production of which conventional aids such as are used.

The pharmaceutical products are preferably produced in and administered in dosage units, each unit comprising a particular dose of the liposome system as active ingredient. In the case of solutions for injection in ampoule form, this dose can be from about 10 mg to about 2000 mg, preferably from about 50 mg to about 2000 mg, with preference from about 250 mg to 500 mg, based on the phospholipid.

Daily doses required for the treatment of an adult patient are, depending on the size of the treated adipose tissue, on administration of solutions for injection, from 5 mg to 500 mg, preferably 250 mg to 500 mg, per injection based on the phospholipid. The solutions for injection can also be diluted before administration, preferably with saline solution. However, in some circumstances, higher or lower daily doses may also be appropriate. The dose also depends on the size of the lipomas, and for small lipomas amounts of from 1 mg to 50 mg, preferably 2 mg to 20 mg, per injection, based on the phospholipid, are entirely sufficient. Administration of the daily dose can take place both through a single dose in the form of a single dosage unit or else a plurality of smaller dosage units and by multiple dosage of divided doses at defined intervals.

The invention is explained in more detail below by means of examples.

### EXAMPLES

### Example 1 Production of the Liposome System

250 g of high-purity soybean phosphatidylcholine which contains more than 90% by weight of phosphatidylcholine, and 126.5 g of deoxycholic acid were dissolved in 1 liter of ethanol. The resulting solution was subsequently evaporated to dryness under reduced pressure. The resulting residue was dispersed in 5 liters of water and then brought by high-pressure homogenization to an average liposome diameter of from 30 nm to 100 nm. The resulting liposome system was then filtered under sterile conditions through a 0.2 μm filter and dispensed under sterile conditions into ampoules each containing 5 ml of liposome system.

### Example 2 Regression of Lipomas

a) The female patient attended for consultation about liposuction of the abdomen, and in the framework of this treatment the following history was taken:

As a child and young adult she was an acrobat and performed movements of the body like those on gymnastic apparatus. During this she suffered a blunt injury with severe effusion of blood underneath the left shoulder blade. Subsequently, especially during particular movements, there was a pronounced raising of the shoulder blade due to a tissue tumor which remained constant over many years.

During the treatment, the question of the therapeutic possibility of removal was then discussed, and removal of the lipoma with the aid of tumescent local anesthesia was recommended to the patient. The subsequently performed partial removal proceeded without difficulty but incompletely. The acute improvement diminished and there was partial regression of the process underneath the shoulder blade, which was then investigated by computed tomography (CT).

The assessment from the radiological findings was as follows: no bony changes in the scapula; in the CT there is suspicion of a distinct residual tissue at the medial underedge of the scapula in the compartment of the trapezius muscle. This muscle is distended and shows accumulations of fat, differential diagnostically a tumor residue. Supplementary NMR tomography recommended in accordance with the above statements, especially if a renewed operation is intended.

Subsequently, when the patient attended again about 4 months after the operation, infiltration with 5 ml of Lipostabil® N i.V. (Rote Liste, March 2003) with a 10 cm-long needle was performed and distributed in this finding. The patient reported slight stinging sensations for one day, but they then disappeared. The lipoma regressed relatively rapidly until symptoms had completely disappeared.

The patient remained free of symptoms thereafter.
b) The male patient had a lipoma about the size of a walnut on the right upper arm. The patient had no disturbances of lipid metabolism, and the serum lipids were in the normal range. An amount of 0.2 ml of Lipostabil®, diluted with 0.2 ml of NaCl ad inj., was injected into the patient. There was clear regression of the lipoma after the first 10 days.

### Example 3 Regression of Pronounced Cellulite

The two female patients had no disturbances of lipid metabolism, and the serum lipids were in the normal range.

Both patients received injections of 0.4 ml of Lipostabil®, diluted with 0.6 ml of NaCl solution inj., (total amount injected 1.0 ml) in one session. 0.1 ml of the solution was injected for each “cellulite mound”, and the total amount was used to treat an area approximately the size of the palm of the hand on the outer sides of both thighs (injection scheme similar to the Botox scheme for hyperhidrosis). With only slight tenderness and sensitivity to touch and moderate erythema there was regression of the raised areas within the first two weeks. A sonographic check was also performed.

1. A method for the treatment of an adipose tissue disorder comprising the administration of an efficacious amount of an aqueous phospholipid system comprising
a) at least one phospholipid,
b) at least one bile acid and
c) water.

2. The method of claim 1, wherein said phospholipid is selected from the group consisting of 3-sn-phosphatidylcholine, soya, 3-sn-phosphatidylcholine, reduced soya, 3-(3sn)-phospohatidyl)glycerol soya, dimyristoylphosphatidylglycerol, lyso-phosphatidylcholine, and dipalmitoylphosphatidylglycerol, or the physiologically tolerated salts thereof, or a mixture thereof.

3. The method of claim 2, wherein the phospholipid is a physiologically tolerated sodium, potassium or ammonium salt.

4. The method of claim 2, wherein the phospholipid comprises soybean phosphatidylcholine.

5. The method of claim 4, wherein the phospholipid comprises at least 90 percent by weight soybean phosphatidylcholine.

6. The method of claim 1, wherein said bile acid is selected from the group consisting of deoxycholic acid, cholic acid, lithocholic acid, chenodeocycholic acid, hyodeoxycholic acid, trihydroxycoprostanic acid, ursodeoxycholic acid, taurocholic acid, and glycocholic acid, or the physiologically tolerated salts thereof, or a mixture thereof.

7. The method of claim 6, wherein the bile acid is a physiologically tolerated sodium, potassium or ammonium salt.

8. The method of claim 1, wherein the mass ratio of phospholipid to bile acid in percent by weight is from 30:1 to 1:0.03.

9. The method of claim 1, wherein the phospholipid concentration is from 0.5% by weight to 30% by weight.

10. The method of claim 1, wherein the adipose tissue disorder is a lipoma, Dercum's disease, Madelung's neck, lipedema, xanthelasama or piezogenic nodules.

11. The method of claim 1, wherein the adipose tissue disorder is adipose tissue tumors.

12. A method for the treatment of cellulite comprising the administration of an efficacious amount of an aqueous phospholipid system comprising
a) at least one phospholipid,
b) at least one bile acid, and
c) water.

13. A method for the treatment of cellulite comprising the administration of an efficacious amount of an aqueous phospholipid system comprising at least one phospholipid or at least one bile acid.
